(19) United States
(12) Patent Application Publication
(10) Pub. No.: US 2026/0253293 A1
(43) Pub. Date: Aug. 27, 2026

(54) METHODS AND APPARATUS FOR DEEP LEARNING BASED ATTENUATION CORRECTION FOR IMAGE RECONSTRUCTION

(71) Applicants: Siemens Medical Solutions USA, Inc., Malvern, PA (US); The Trustees of The University of Pennsylvania, Philadelphia, PA (US)

(72) Inventors: Joshua Schaefferkoetter, Knoxville, TN (US); Vladimir Panin, Knoxville, TN (US); Deepak Bharkhada, Knoxville, TN (US); Mael Millardet, Knoxville, TN (US)

(21) Appl. No.: 19/059,620

(22) Filed: Feb. 21, 2025

(51) Int. Cl. G06T 11/00; G06T 7/00

(52) U.S. Cl. CPC G06T 12/20; G06T 7/97; G06T 2207/10081; G06T 2207/10104; G06T 2207/20084

(57) ABSTRACT

Systems and methods for generating registered attenuation maps are disclosed. For example, positron emission tomography (PET) measurement data, and co-modality measurement data from an anatomy modality, such as computed tomography (CT) data, is received from an image scanning system. A histo-image is generated based on the PET measurement data, and a co-modality image is generated based on the co-modality measurement data. A trained machine learning process is applied to the histo-image and the co-modality image. The trained machine learning process is configured to correct for misalignment between the histo-image and the co-modality image. Based on the application of the trained machine learning process to the histo-image and the co-modality image, a registered attenuation map is generated. In some examples, a PET image is reconstructed using the registered attenuation map.

### FIELD

Aspects of the present disclosure relate in general to medical diagnostic systems and, more particularly, to reconstructing images from nuclear imaging systems for diagnostic and reporting purposes.

### BACKGROUND

Nuclear imaging systems can employ various technologies to capture images. For example, some nuclear imaging systems employ positron emission tomography (PET) to capture images. PET is a nuclear medicine imaging technique that produces tomographic images representing the distribution of positron emitting isotopes within a body. Some nuclear imaging systems employ computed tomography (CT), for example, as a co-modality. CT is an imaging technique that uses x-rays to produce anatomical images. Magnetic Resonance Imaging (MRI) is an imaging technique that uses magnetic fields and radio waves to generate anatomical and functional images, and can also be used as a co-modality. Typically, these nuclear imaging systems capture measurement data, and process the captured measurement data using mathematical algorithms to reconstruct medical images. Some nuclear imaging systems combine images from PET and CT scanners during an image fusion process to produce images that show information from both a PET scan and a CT scan (e.g., PET/CT systems). For these PET/CT systems, the CT measurement information can be used to correct the PET measurement data for attenuation (i.e., attenuation correction of the PET image). Similarly, some nuclear imaging systems combine images from PET and MRI scanners to produce images that show information from both a PET scan and an MRI scan.

These conventional systems, however, can suffer from drawbacks. For instance, subjects may move during and/or between PET and CT scans, thereby causing misalignment between the captured PET measurement data and CT measurement data. This misalignment can lead to inaccurate attenuation correction, for instance, when the CT measurement data is used to correct the PET measurement data for attenuation. Moreover, current motion correction techniques can be time-intensive and cause inaccurate and lower quality medical images. As such, there are opportunities to address deficiencies in nuclear imaging systems.

### SUMMARY

Systems and methods for generating registered attenuation maps based on deep learning-based processes are disclosed.

In some embodiments, a computer-implemented method includes receiving positron emission tomography (PET) measurement data (e.g., TOF sinogram data, list mode data) from an image scanning system. The method also includes receiving co-modality measurement data from the image scanning system. Further, the method includes generating a histo-image based on the PET measurement data. The method also includes generating a co-modality image based on the co-modality measurement data. The method further includes applying a trained machine learning process to the histo-image and the co-modality image and, based on the application of the trained machine learning process to the histo-image and the co-modality image, generating registered attenuation map data characterizing a registered attenuation map. The method also includes storing the registered attenuation map data in a data repository.

In some embodiments, a non-transitory computer readable medium stores instructions that, when executed by at least one processor, cause the at least one processor to perform operations. The operations include receiving PET measurement data from an image scanning system. The operations also include receiving co-modality measurement data from the image scanning system. Further, the operations include generating a histo-image based on the PET measurement data. The operations also include generating a co-modality image based on the co-modality measurement data. The operations further include applying a trained machine learning process to the histo-image and the co-modality image and, based on the application of the trained machine learning process to the histo-image and the co-modality image, generating registered attenuation map data characterizing a registered attenuation map. The operations also include storing the registered attenuation map data in a data repository.

In some embodiments, an apparatus includes a memory storing instructions, and at least one processor communicatively coupled the memory. The at least one processor is configured to execute the instructions to perform operations. The operations include receiving PET measurement data from an image scanning system. The operations also include receiving co-modality measurement data from the image scanning system. Further, the operations include generating a histo-image based on the PET measurement data. The operations also include generating a co-modality image based on the co-modality measurement data. The operations further include applying a trained machine learning process to the histo-image and the co-modality image and, based on the application of the trained machine learning process to the histo-image and the co-modality image, generating registered attenuation map data characterizing a registered attenuation map. The operations also include storing the registered attenuation map data in a data repository.

In some embodiments, a computer-implemented method includes receiving histo-images and corresponding co-modality images. The method also includes inputting the histo-images and the co-modality images into a machine learning process and, based on inputting the histo-images and the co-modality images, generating output data characterizing attenuation maps. Further, the method includes adjusting weights of the machine learning process based on the output data, wherein the weight adjustment corrects for misalignments between the histo-images and the co-modality images. The method also includes generating a loss value based on the output data and ground truth data. The method further includes determining the machine learning process is trained based on the loss value and a threshold value. The method also includes storing parameters of the trained machine learning process in a data repository.

In some embodiments, a non-transitory computer readable medium stores instructions that, when executed by at least one processor, cause the at least one processor to perform operations. The operations include includes receiving histo-images and corresponding co-modality images. The operations also include inputting the histo-images and the co-modality images into a machine learning process and, based on inputting the histo-images and the co-modality images, generating output data characterizing attenuation maps. Further, the operations include adjusting weights of the machine learning process based on the output data, wherein the weight adjustment corrects for misalignments between the histo-images and the co-modality images. The operations also include generating a loss value based on the output data and ground truth data. The operations further include determining the machine learning process is trained based on the loss value and a threshold value. The operations also include storing parameters of the trained machine learning process in a data repository.

In some embodiments, an apparatus includes a memory storing instructions, and at least one processor communicatively coupled the memory. The at least one processor is configured to execute the instructions to perform operations. The operations include includes receiving histo-images and corresponding co-modality images. The operations also include inputting the histo-images and the co-modality images into a machine learning process and, based on inputting the histo-images and the co-modality images, generating output data characterizing attenuation maps. Further, the operations include adjusting weights of the machine learning process based on the output data, wherein the weight adjustment corrects for misalignments between the histo-images and the co-modality images. The operations also include generating a loss value based on the output data and ground truth data. The operations further include determining the machine learning process is trained based on the loss value and a threshold value. The operations also include storing parameters of the trained machine learning process in a data repository.

### BRIEF DESCRIPTION OF THE DRAWINGS

The following will be apparent from elements of the figures, which are provided for illustrative purposes and are not necessarily drawn to scale.

FIG. 1 illustrates a nuclear image reconstruction system, in accordance with some embodiments.

FIG. 2 illustrates a block diagram of an example computing device that can perform one or more of the functions described herein, in accordance with some embodiments.

FIG. 3 illustrates a neural network of a nuclear imaging system, in accordance with some embodiments.

FIG. 4 illustrates a machine learning model training system, in accordance with some embodiments.

FIG. 5 is a flowchart of an example method to generate a registered attenuation map, in accordance with some embodiments.

FIG. 6 is a flowchart of an example method to train a machine learning model, in accordance with some embodiments.

FIG. 7 illustrates attenuation corrected nuclear images, in accordance with some embodiments.

### DETAILED DESCRIPTION

This description of the exemplary embodiments is intended to be read in connection with the accompanying drawings, which are to be considered part of the entire written description. Independent of the grammatical term usage, individuals with male, female, or other gender identities are included within the term.

The exemplary embodiments are described with respect to the claimed systems as well as with respect to the claimed methods. Furthermore, the exemplary embodiments are described with respect to methods and systems for image reconstruction, as well as with respect to methods and systems for training functions used for image reconstruction. Features, advantages, or alternative embodiments herein can be assigned to the other claimed objects and vice versa. For example, claims for the providing systems can be improved with features described or claimed in the context of the methods, and vice versa. In addition, the functional features of described or claimed methods are embodied by objective units of a providing system. Similarly, claims for methods and systems for training image reconstruction functions can be improved with features described or claimed in context of the methods and systems for image reconstruction, and vice versa.

Various embodiments of the present disclosure can employ machine learning methods or processes to provide clinical information from nuclear imaging systems. For example, the embodiments can employ machine learning methods or processes to reconstruct images based on captured measurement data, and provide the reconstructed images for clinical diagnosis. In some embodiments, machine learning methods or processes are trained, to improve the reconstruction of images.

Quantitative positron emission tomography (PET) or single-photon emission computed tomography (SPECT) generally requires an attenuation map (e.g., mu-map) to correct for a number of photons that have either been lost for a sinogram bin (i.e., attenuation correction) or wrongly assigned to another sinogram bin (i.e., scatter correction). The corrections generally depend on an accurate knowledge of photon values within a subject. The attenuation map characterizing the corrections (e.g., μ-map) can be calculated or estimated using an accompanying anatomical modality, such as computed tomography (CT) or magnetic resonance (MR). Subjects, however, may move during image capturing, which can cause misalignment issues during PET reconstruction. Motion of the subject during or between consecutive scans can result in a μ-map that is spatially mismatched from the captured PET measurement data. As a result, when used for attenuation correction, the misaligned μ-map can introduce quantitative artifacts in reconstructed PET images. For instance, subjects may breathe or voluntarily move portions of their body between consecutive scans. The movement may result in improper image alignment and thus improper attenuation correction of the PET measurement data. Indeed, greater mismatch and improper attenuation correction may be experienced with longer scans. The misalignment and improper attenuation correction may cause inaccuracies in the reconstructed images that may be displayed to medical professionals for diagnostic purposes.

In some embodiments, a machine learning model, such as a neural network, is trained using PET histo-images and corresponding co-modality images (e.g., CT images, MRI images) to generate registered attenuation maps (e.g., μ-maps). For instance, during the training, pairs of PET histo-images and co-modality images, and ground truth data characterizing a misalignment between the pairs of PET histo-images and co-modality images, is inputted into the machine learning model. In some examples, to generate the pairs PET histo-images and co-modality images, otherwise aligned PET histo-images and co-modality images are purposely adjusted to be misaligned, and ground truth data is generated characterizing the misalignment. The training may adjust weights of the machine learning model such that the trained machine learning model is configured to generate registered attenuation maps that are corrected for misalignment (e.g., anatomical misalignment) between the pairs of PET histo-images and co-modality images. A generated registered attenuation map can include an attenuation correction value (e.g., a linear attenuation coefficient) for each pixel location of the PET histo-image, where the attenuation values can be used to correct the corresponding PET-histo-image values for attenuation. For instance, to generate the registered attenuation map, the trained machine learning model may adjust a three-dimensional position of each attenuation correction value obtained from the co-modality image to align to a corresponding pixel location in the PET histo-image. As a result, the generated registered attenuation map may include attenuation correction values associated with anatomical features in the co-modality image that are more aligned with the corresponding anatomical features in the PET histo-image. In some embodiments, a PET image can be using the generated registered attenuation map, where the reconstructed PET image can include more accurate attenuation corrections. For instance, the PET image can be reconstructed based on applying a trained machine learning process or artificial process (e.g., Fast PET) to the PET histo-image and the generated registered attenuation map.

Among other advantages, the embodiments can more accurately generate attenuation maps from varying modalities (e.g., PET and CT or PET and MRI), such as in cases where a subject moves during scans. Further, the embodiments may reduce various types of attenuation correction artifacts in reconstructed PET images of subjects that move during scanning. The embodiments may also reduce associated diagnostic errors, and provide higher quality attenuation and scatter corrections leading to more reliable PET quantification. Persons of ordinary skill in the art may recognize these and other advantages as well.

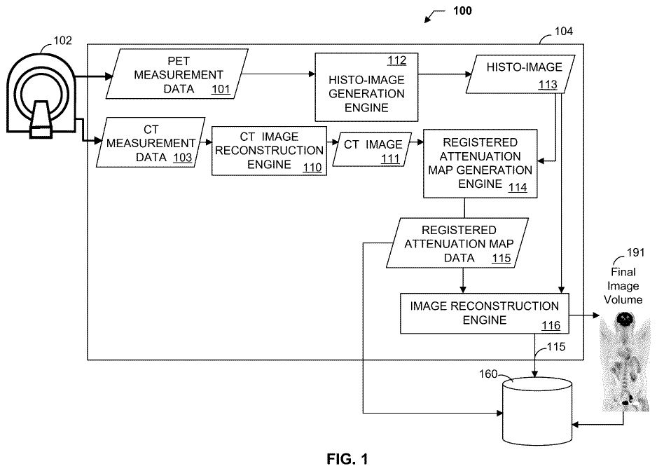

FIG. 1 illustrates an embodiment of a nuclear imaging system 100. As illustrated, nuclear imaging system 100 includes image scanning system 102 and image reconstruction system 104. Image scanning system 102 can be, for instance, a PET/CT scanner that can capture PET and CT images. In other examples, the image scanning system 102 can be a PET/MR system, for instance.

In this example, image scanning system 102 can scan a subject to capture CT images, and can generate CT measurement data 103 characterizing the CT scans. Image scanning system 102 can also capture PET images (e.g., of the person), and generate PET measurement data 101 (e.g., PET raw data, such as sinogram data or list mode data) based on the captured PET images. The PET measurement data 101 can represent anything imaged in the scanner's field-of-view (FOV) containing positron emitting isotopes. For example, the PET measurement data 101 can represent whole-body image scans, such as image scans from a patient's head to thigh. Image scanning system 102 can transmit the CT measurement data 103 and the PET measurement data 101 to image reconstruction system 104.

Image reconstruction system 104 includes CT image reconstruction engine 110, histo-image generation engine 112, registered attenuation map generation engine 114, and image reconstruction engine 116. In some examples, all or parts of CT image reconstruction engine 110, image reconstruction system 104, including each of histo-image generation engine 112, registered attenuation map generation engine 114, and image reconstruction engine 116, are implemented in hardware, such as in one or more field-programmable gate arrays (FPGAs), one or more application-specific integrated circuits (ASICs), one or more state machines, one or more computing devices, digital circuitry, or any other suitable circuitry. In some examples, parts or all of image reconstruction system 104 can be implemented in software as executable instructions such that, when executed by one or more processors, cause the one or more processors to perform respective functions as described herein. The instructions can be stored in a non-transitory, computer-readable storage medium, for instance.

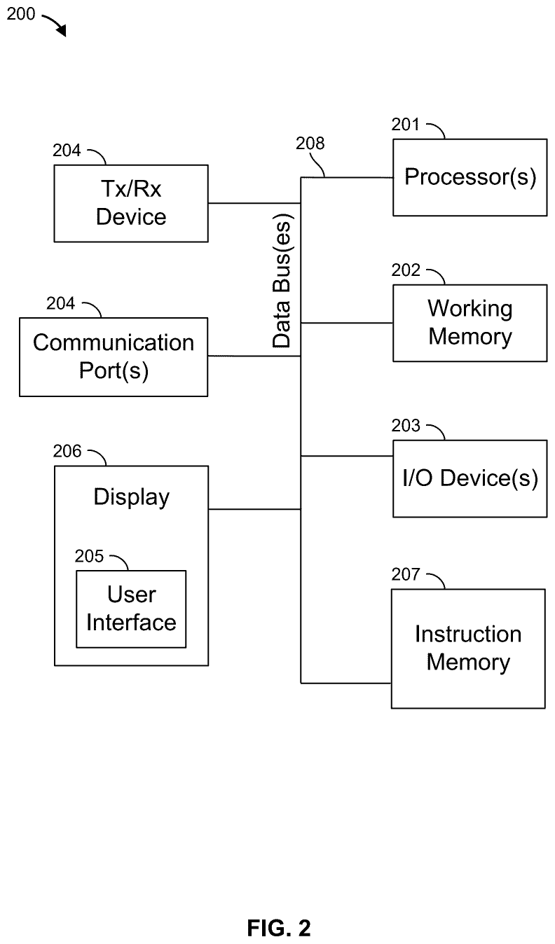

For example, FIG. 2 illustrates a computing device 200 that can be employed by the image reconstruction system 104. Computing device 200 can implement, for example, one or more of the functions of image reconstruction system 104 described herein.

Computing device 200 can include one or more processors 201, working memory 202, one or more input/output devices 203, instruction memory 207, a transceiver 204, one or more communication ports 209, and a display 206, all operatively coupled to one or more data buses 208. Data buses 208 allow for communication among the various devices. Data buses 208 can include wired, or wireless, communication channels.

Processors 201 can include one or more distinct processors, each having one or more cores. Each of the distinct processors can have the same or different structure. Processors 201 can include one or more central processing units (CPUs), one or more graphics processing units (GPUs), application specific integrated circuits (ASICs), digital signal processors (DSPs), and the like. Processors 201 can be configured to perform a certain function or operation by executing code, stored on instruction memory 207, embodying the function or operation. For example, processors 201 can be configured to perform one or more of any function, method, or operation disclosed herein by executing instructions stored in instruction memory 207.

Instruction memory 207 can store instructions that can be accessed (e.g., read) and executed by processors 201. For example, instruction memory 207 can be a non-transitory, computer-readable storage medium such as a read-only memory (ROM), an electrically erasable programmable read-only memory (EEPROM), flash memory, a removable disk, CD-ROM, any non-volatile memory, or any other suitable memory. For example, instruction memory 207 can store instructions that, when executed by the one or more processors 201, cause one or more processors 201 to perform one or more of the functions of image reconstruction system 104, such as one or more of the functions of any of CT image reconstruction engine 110, histo-image generation engine 112, registered attenuation map generation engine 114, and image reconstruction engine 116, described herein.

Processors 201 can store data to, and read data from, working memory 202. For example, processors 201 can store a working set of instructions to working memory 202, such as instructions loaded from instruction memory 207. Processors 201 can also use working memory 202 to store dynamic data created during the operation of computing device 200. Working memory 202 can be a random access memory (RAM) such as a static random access memory (SRAM) or dynamic random access memory (DRAM), or any other suitable memory.

Input/output devices 203 can include any suitable device that allows for data input or output. For example, input-output devices 203 can include one or more of a keyboard, a touchpad, a mouse, a stylus, a touchscreen, a physical button, a speaker, a microphone, or any other suitable input or output device.

Communication port(s) 209 can include, for example, a serial port such as a universal asynchronous receiver/transmitter (UART) connection, a Universal Serial Bus (USB) connection, or any other suitable communication port or connection. In some examples, communication port(s) 207 allows for the programming of executable instructions in instruction memory 207. In some examples, communication port(s) 207 allow for the transfer (e.g., uploading or downloading) of data, such as CT measurement data 103 and histo-images 113 described further herein.

Display 206 can display user interface 205. User interfaces 205 can enable user interaction with computing device 200. For example, user interface 205 can be a user interface for an application that allows for the viewing of the final image volumes 191 described further herein. In some examples, a user can interact with user interface 205 by engaging (e.g., touching with a finger or stylus) an input/output device 203. In some examples, display 206 can be a touchscreen, where user interface 205 is displayed on the touchscreen.

Transceiver 204 allows for communication with a network, such as a Wi-Fi® network, an Ethernet network, a cellular network, or any other suitable communication network. For example, if operating in a Wi-Fi® network, transceiver 204 is configured to allow communications with the Wi-Fi® network (e.g., and, in some examples, other devices on the Internet to which the Wi-Fi® network is connected). Processor(s) 201 is operable to receive data from, or send data to, a network via transceiver 204.

Referring back to FIG. 1, CT image reconstruction engine 110 receives CT measurement data 103 (e.g., CT raw data) and, based on the CT measurement data 103, generates reconstructed CT image 111. CT image reconstruction engine 110 can generate reconstructed CT images 111 based on corresponding CT measurement data 103 using any suitable method known in the art. In addition, histo-image generation engine 112 receives PET measurement data 101. Based on the receives PET measurement data 101, the histo-image generation engine 112 generates histo-images 113 (e.g., multi-view histo-images). The histo-image generation engine 112 can generate the histo-images based on any suitable method known in the art. For instance, the histo-image generation engine 112 can generate a histo-image 113 based on applying a time-of-flight (TOF) back-projection process to TOF sinograms (as characterized by the PET measurement data 101), or applying a back-projection process to each event of list mode data (as characterized by the PET measurement data 101). In some examples, the histo-image generation engine 112 can be a most likely annihilation position histogrammer that places photon coincidences into a histo-image representation, e.g., histo-image 113. In some examples, the histo-image generation engine 112 can be a nearest-neighbor histogrammer that applies a nearest neighbor approach to the list mode data to generate the histo-image 113.

Further, registered attenuation map generation engine 114 receives the CT images 111 and the histo-images 113, and applies a machine learning process to the CT images 111 and the histo-images 113 to generate registered attenuation map data 115 characterizing a registered attenuation map (e.g., μ-map). As described further herein, the registered attenuation map data 115 can include attenuation correction values (e.g., a linear attenuation coefficients), where each attenuation correction value is associated with a position (e.g., 3D position) that has been adjusted based on detected misalignments between a histo-image 113 and corresponding CT image 111. For instance, the registered attenuation map generation engine 114 may determine an alignment of a CT image 111 to a histo-image 113, and may generate the registered attenuation map data 115 based on the alignment. The registered attenuation map generation engine 114 can include attenuation correction values at corresponding positions that have been aligned to corresponding pixels of the histo-image 113.

As described herein, to generate the registered attenuation map data 115, the registered attenuation map generation engine 114 may apply a trained machine learning process to a received CT image 111 and corresponding histo-image 113. For instance, the registered attenuation map generation engine 114 may generate CT feature vectors based on the CT image 111, and histo-image feature vectors based on the histo-image 113. Further, the registered attenuation map generation engine 114 may input the CT feature vectors and the histo-image feature vectors into a trained machine learning model and, based on inputting the CT feature vectors and the histo-image feature vectors into the trained machine learning model, generates the registered attenuation map data 115. In some instances, the trained machine learning model may be a trained neural network, such as a convolutional neural network.

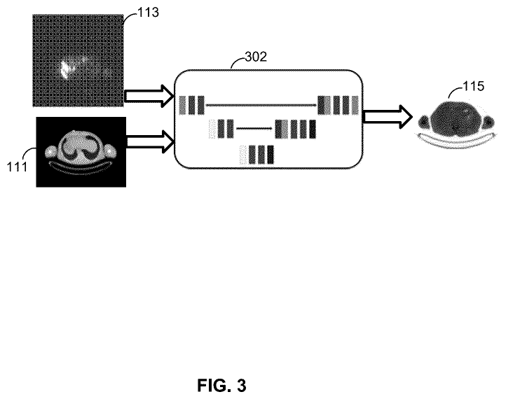

FIG. 3, for example, illustrates an example of the functionality of the registered attenuation map generation engine 114. In this example, a histo-image 113 and a CT image 111 (e.g., an anatomical image) is inputted to a trained neural network 302. The trained neural network 302 may be configured to adjust positions of the pixels of the CT image 111 to align with corresponding pixels of the histo-image 113, and to generate registered attenuation map data 115 characterizing a registered attenuation map based on the adjusted pixel positions. The trained neural network 302 can generate the registered attenuation map that at least reduces, if not avoids, motion mis-registration. In some instances, the trained neural network 302 may be based on a U-NET style architecture. For example, the trained neural network 302 may be based on the U-NET neural network described in “FastPET: Near Real-Time PET Reconstruction from Histo-Images Using a Neural Network,” by Whiteley et al., 15 Jun. 2020.¹ ¹ Available at https://arxiv.org/abs/2002.04665 (last accessed on Nov. 21, 2024).

Referring back to FIG. 1, in some examples, the registered attenuation map generation engine 114 may store the registered attenuation map 115 in a data repository 160. Further, in some examples, the image reconstruction engine 116 receives the registered attenuation map data 115 and the histo-image 113, and generates a final image volume 191. For instance, the image reconstruction engine 116 may apply a trained reconstruction machine learning process to the histo-image 113 and the registered attenuation map data 115 to generate the final image volume 191. In some examples, the trained reconstruction machine learning process includes inputting the histo-image 113 and the registered attenuation map data 115 to a trained reconstruction neural network, where the trained reconstruction neural network generates the final image volume 191 based on the inputted histo-image 113 and the registered attenuation map data 115. In some instances, the image reconstruction engine 116 applies an artificial intelligence process, such as a Fast PET process, to the histo-image 113 and the registered attenuation map data 115 to generate the final image volume 191. The image reconstruction engine 116 may then store one or more of the registered attenuation map data 115 and the final image volume 191 in the data repository 160.

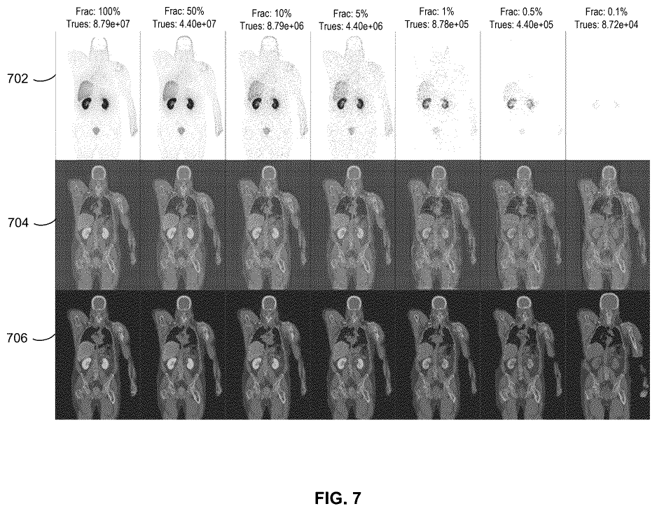

FIG. 7 illustrates images associated with motion estimation and subsequent registration as described herein. Referring to FIG. 7, a first row 702 of various PET images are illustrated, where the PET images include decreasing PET counts (e.g., due to increasing noise) from left to right. Below the first row 702 is a second row 704 that includes the PET images overlaid with corresponding CT images, and grids representing motion estimated by a registration algorithm, such as motion that can be estimated by the registered attenuation map generation engine 114 between the histo-image 113 and the CT image 111. Further, a third row 706 includes PET images overlaid with the registered CT images using the estimated motions (e.g., PET images aligned with the CT images based on the estimated motion). As illustrated, the third row 706 illustrates an alignment between the PET images and the CT images. These images indicate a performance of CT-to-PET registration in accordance with the embodiments described herein for various levels of PET noise. The noise levels are represented as fractions of the original PET data (and also as total true counts). As can be seen, the registration is well-behaved at relatively high levels of noise.

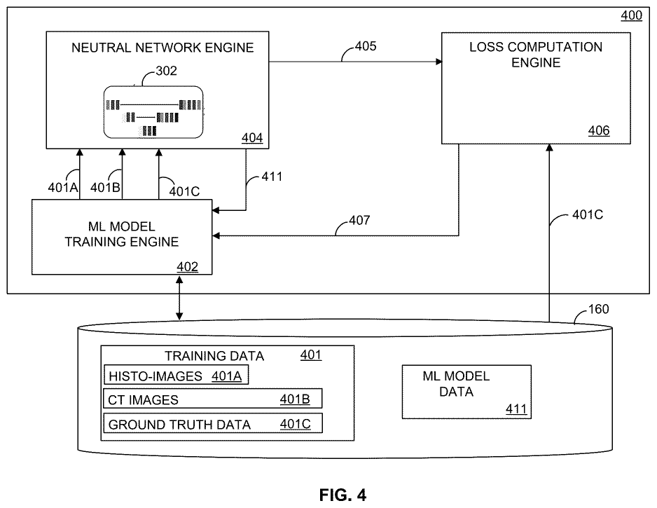

FIG. 4 illustrates a machine learning model training system 400 that includes a machine learning (ML) model training engine 402, a neural network engine 404, and a loss computation engine 406. In some examples, all or parts of the machine learning model training system 400 are implemented in hardware, such as in one or more FPGAs, one or more ASICs, one or more state machines, one or more computing devices, digital circuitry, or any other suitable circuitry. In some examples, parts or all of the machine learning model training system 400 can be implemented in software as executable instructions such that, when executed by one or more processors, cause the one or more processors to perform respective functions as described herein. For instance, in some examples, processors 201 of computing device 200 can execute instructions stored in instruction memory 207 to carry out one or more of the functions of any of the model training engine 402, the neural network engine 404, and the loss computation engine 406.

As illustrated, data repository 160 includes training data 401 that can be used to train a machine learning model to generate attenuation maps that align to measurement data, such as PET or SPECT measurement data. The training data 401 includes histo-images 401A and corresponding CT images 401B. Each pair of histo-images 401A and CT images 401B may be based on consecutive scans of a patient that may have moved between or during the scans. In addition, the training data 401 includes ground truth data 401C characterizing a misalignment between corresponding pairs of histo-images 401A and CT images 401B. For instance, the ground truth data 401C may include a 3D displacement vector (e.g., characterizing a displacement in each of an X, Y, and Z direction) between corresponding pixels of each pair of histo-images 401A and CT images 401B.

In some instances, to generate misaligned pairs of histo-images 401A and CT images 401B, the ML model training engine 402 may generate an adjusted histo-image 401A by adjusting pixel locations of what is otherwise a histo-image that is aligned to a CT image 401B (e.g., purposeful misalignment). Based on the adjustment, the ML model training engine 402 generates corresponding ground truth data 401C (i.e., the ground truth data 401C characterizes the misalignment) for the adjusted histo-image 401A and the CT image 401B. Similarly, in some instances, the ML model training engine 402 may generate an adjusted CT image 401B by adjusting pixel locations of what is otherwise a CT image that is aligned to a histo-image 401A. Based on the adjustment, the ML model training engine 402 generates corresponding ground truth data 401C for the adjusted CT image 401B and the histo-image 401A.

To train the neural network 302, the ML model training engine 402 receives, from the data repository 160, one or more epochs of training data 401 that include corresponding pairs of histo-images 401A and CT images 401B and ground truth data 401C, and transmits the pairs of histo-images 401A and CT images 401B and ground truth data 401C to the neural network engine 404. Further, the neural network engine 404 inputs the pairs of histo-images 401A and CT images 401B to the neural network 302. For instance, the neural network engine 404 may generate histo-image feature vectors based on the histo-images 401A, and CT feature vectors based on the CT images 401B. The neural network engine 404 may input the histo-image feature vectors and the CT feature vectors to the neural network 302 and, based on the inputted histo-image feature vectors and the CT feature vectors, the neural network 302 may generate output data 405 characterizing an attenuation map. The attenuation map can account for pixel position displacements.

Further, the neural network engine 404 can adjust one or more weights of the neural network 302 during the training. For instance, the neural network engine 404 can compute a loss based on the output data 405 and the ground truth data 401C, and can adjust the one or more weights based on the computed loss. The loss function can be, for example, a mean squared error (MSE) loss function, or any other suitable loss function. In some instances, the neural network engine 404 attempts to minimize an objective function that operates on the output data 405 and the ground truth data 401C. The objective function may include a comparison (e.g., a difference) between pixel positions (e.g., 3D pixel positions) of the attenuation map (characterized by the output data 405), and the pixel positions (e.g., 3D pixel positions) of the CT image 401B as adjusted by the pixel displacements (characterized by the ground truth data 401C). Based on the output of the objective function, the neural network engine 404 may update the weights of the neural network 302. For instance, the neural network engine 404 may adjust the weights based on a learning rate constraint of the objective function.

Further, the loss computation engine 406 may receive the output data 405 from the neural network engine 404, and the ground truth data 401C from the data repository 160. The loss computation engine 406 may determine a loss value 407 based on the output data 405 and the ground truth data 401C. For example, the loss computation engine 406 may compute the loss value 407 based on a loss function. The loss value 407 can be computed using any suitable loss function (e.g., image reconstruction loss function), such as any of the mean square error (MSE), mean absolute error (MAE), binary cross-entropy (BCE), Sobel, Laplacian, and Focal binary loss functions. The loss computation engine 406 can transmit the loss value 407 to the ML model training engine 402.

Based on the loss value 407, the ML model training engine 402 can determine whether the neural network 302 is trained. For instance, if the loss value 407 at least meets (e.g., exceeds, is below) a corresponding loss threshold, then the ML model training engine 402 determines that the neural network 302 is trained. The ML model training engine 402 may obtain ML model data 411 associated with the trained neural network 302 from the neural network engine 404, and may store the ML model data 411 within the data repository 160. The ML model data 411 can include parameters of the trained neural network 302, such as weights, hyperparameters, constraint values, and coefficients. The trained neural network 302 can be established (e.g., configured and executed) based on the ML model data 411.

Otherwise, if the loss value 407 does not at least meet the loss threshold, the ML model training engine 402 determines that the neural network 302 is not trained. In this case, the ML model training engine 402 continues to transmit histo-images 401A and CT images 401B (e.g., epochs of histo-images 401A and CT images 401B) to the neural network engine 404 to continue training the neural network 302. The training of the neural network 302 may continue until the loss value 407 at least meets the loss threshold.

In some instances, once the loss at least meets the loss threshold, the neural network 302 may be validated using previously unused histo-images 401A and CT images 401B. For example, the ML model training engine 402 may transmit additional histo-images 401A and CT images 401B to the neural network engine 404 for inputting to the neural network 302. In response, the neural network 302 generates additional output data 405 characterizing registered attenuation maps. The loss computation engine 406 can receive the loss value 407, and compute a loss value 407 based on the additional output data 405 and additional ground truth data 401C. The loss computation engine 406 can transmit the loss value 407 to the ML model training engine 402. The ML model training engine 402 can determine whether the neural network 302 is validated based on the loss value 407. For example, if the loss value 407 at least meets (e.g., exceeds, is below) a corresponding loss threshold, then the ML model training engine 402 determines that the neural network 302 is validated. The ML model training engine 402 may then obtain the ML model data 411 from the neural network engine 404, and may store the ML model data 411 within the data repository 160. Otherwise, if the loss value 407 does not at least meet the loss threshold, the ML model training engine 402 determines that the neural network 302 is not validated, and may continue to train and validate the neural network 302 as described herein.

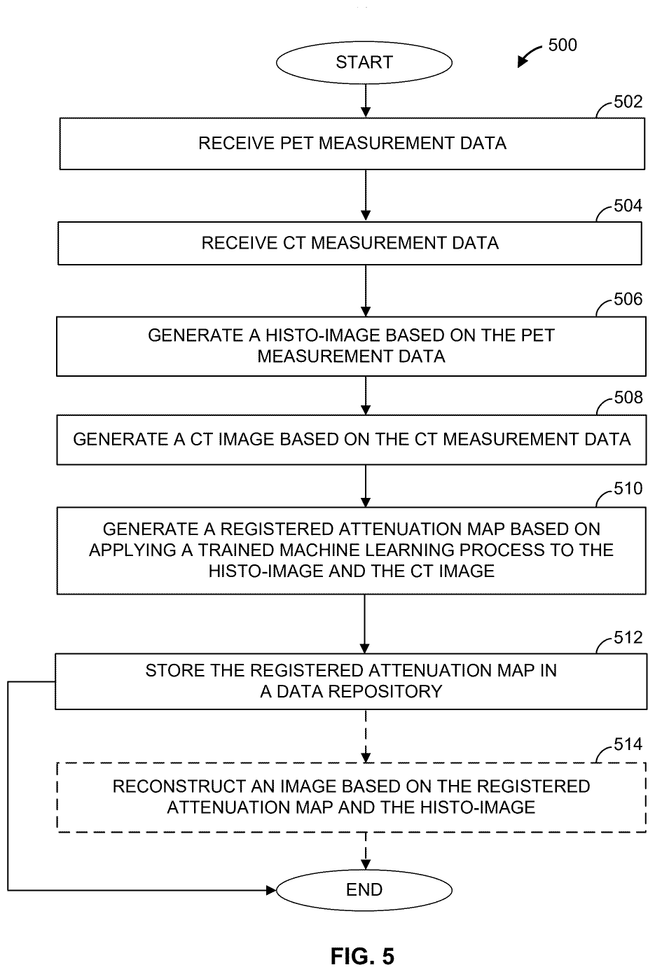

FIG. 5 is a flowchart of an example method 500 to generate a registered attenuation map. The method can be performed by, for example, the image reconstruction system 104.

Beginning at block 502, PET measurement data is received. For instance, image reconstruction system 104 may receive PET measurement data 101 from image scanning system 102. At block 504, CT measurement data is received. For example, the image reconstruction system 104 may receive CT measurement data 103 from the image scanning system 102. Proceeding to block 506, a histo-image is generated based on the PET measurement data. For example, the image reconstruction system 104 may apply a back-projection process to the PET measurement data to generate the histo-image. At block 508, a CT image is generated based on the CT measurement data. For instance, the image reconstruction system 104 may reconstruct a CT image based on the CT measurement data 103 using any suitable method known in the art.

Further, and at block 510, a registered attenuation map is generated based on applying a trained machine learning process to the histo-image and the CT image. For example, the image reconstruction system 104 may generate first feature vectors based on the histo-image, and second feature vectors based on the CT image. The image reconstruction system 104 may input the first feature vectors and second feature vectors to a trained neural network, such as the trained neural network 302. Based on the inputted feature vectors, the trained neural network generates output data characterizing a registered attenuation map, such as registered attenuation map data 115. As described herein, the trained neural network is configured to generate an attenuation map is this corrected for misalignment between the histo-image and the CT image.

At block 512, the registered attenuation map is stored in a data repository. For instance, the image reconstruction system 104 may store the registered attenuation map data 115 in data repository 160. In some examples, at block 514, an image is reconstructed based on the generated registered attenuation map. For example, the image reconstruction system 104 may reconstruct the final image volume 191 based on the registered attenuation map data 115 and the histo-image 113. The image reconstruction system 104 may then provide the final image volume for display (e.g., for displaying on display 206).

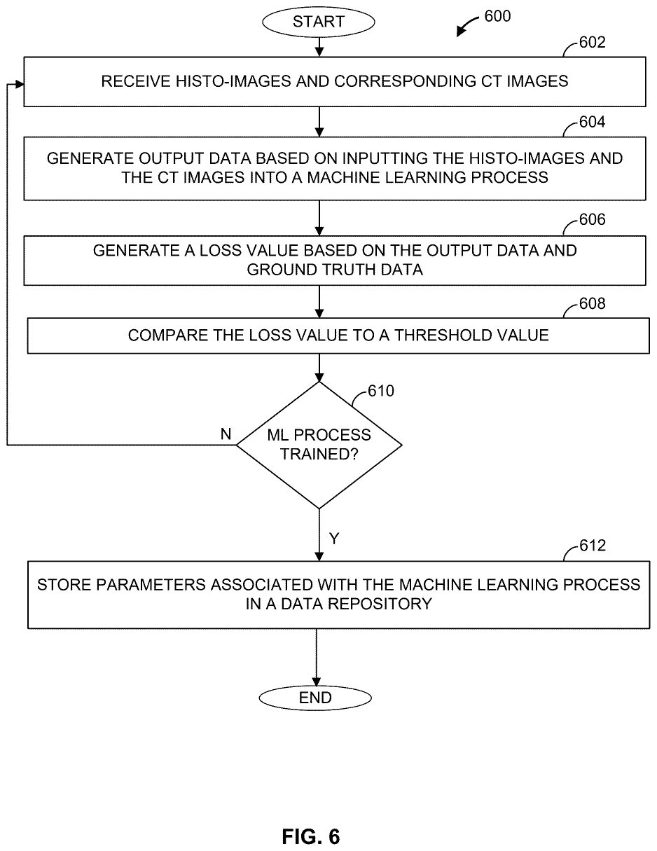

FIG. 6 is a flowchart of an example method 600 to train a machine learning model, such as the neural network 302. The method can be performed by, for example, the machine learning model training system 400.

Beginning at block 602, histo-images and corresponding CT images are received. For example, the machine learning model training system 400 may obtain, from data repository 160, histo-images 401A and CT images 401B. At block 604, the histo-images and CT images are input to a machine learning process and, based on inputting the histo-images and CT images, output data is generated. For instance, as described herein, the machine learning model training system 400 may input a histo-image 113 and a CT image 111 into the neural network 302 and, based on inputting the histo-image 113 and the CT image 111, generates the output data 405.

Further, at block 606, a loss value is generated based on the output data and ground truth data, where the ground truth data characterizes a misalignment between pairs of the histo-image and CT image. The machine learning model training system 400 may generate the loss value 407 based on comparing the output data 405 with the ground truth data 401C. For example, the loss computation engine 406 may compute the loss value 407 based on applying a loss function to the output data 405 and the ground truth data 401C. Proceeding to block 608, the loss value is compared to a threshold value. For example, the machine learning model training system 400 can determine if the loss value 407 at least meets the corresponding threshold value.

At block 610, and based on the comparison, a determination is made as to whether the machine learning process is trained. For example, the machine learning model training system 400 may determine that the neural network 302 is trained when the loss value 407 at least meets (e.g., is at or above) the corresponding threshold value. The machine learning model training system 400 may determine, however, that the neural network 302 is not trained when the loss value 407 does not meet (e.g., is below) the corresponding threshold value. If the machine learning process is not trained, the method proceeds back to block 602 to continue with training the machine learning process. Otherwise, if the machine learning process is trained, the method proceeds to block 612.

At block 612, parameters associated with the machine learning process is stored in a data repository. The parameters characterize the trained machine learning model. For example, the parameters can include, for instance, weights, hyperparameters, constraint values, and coefficients. For instance, as described herein, the machine learning model training system 400 may obtain ML model data 411 associated with the trained neural network 302, and may store the ML model data 411 within the data repository 160. The trained neural network 302 can be established (e.g., configured and executed) based on the stored parameters.

The following is a list of non-limiting illustrative embodiments disclosed herein:

### Illustrative Embodiment 1

A computer-implemented method comprising:
receiving positron emission tomography (PET) measurement data from an image scanning system;
receiving co-modality measurement data from the image scanning system;
generating a histo-image based on the PET measurement data;
generating a co-modality image based on the co-modality measurement data;
applying a trained machine learning process to the histo-image and the co-modality image and, based on the application of the trained machine learning process to the histo-image and the co-modality image, generating registered attenuation map data characterizing a registered attenuation map; and
storing the registered attenuation map data in a data repository.

### Illustrative Embodiment 2

The computer-implemented method of illustrative embodiment 1, wherein applying the trained machine learning process to the histo-image and the co-modality image comprises inputting the histo-image and the co-modality image to a trained neural network.

### Illustrative Embodiment 3

The computer-implemented method of illustrative embodiment 2, further comprising:
generating first feature vectors based on the histo-image;
generating second feature vectors based on the co-modality image; and
inputting the first feature vectors and the second feature vectors into the trained neural network.

### Illustrative Embodiment 4

The computer-implemented method of any of illustrative embodiments 1-3, further comprising reconstructing a PET image based on the registered attenuation map data and the histo-image.

### Illustrative Embodiment 5

The computer-implemented method of illustrative embodiment 4, further comprising providing the PET image for display.

### Illustrative Embodiment 6

The computer-implemented method of any of illustrative embodiments 1-5, wherein the trained machine learning process generates the registered attenuation map data based on detecting a misalignment between the histo-image and the co-modality image.

### Illustrative Embodiment 7

The computer-implemented method of illustrative embodiment 6, wherein the detected misalignment is between pixel locations of the histo-image and pixel locations the co-modality image.

### Illustrative Embodiment 8

The computer-implemented method of any of illustrative embodiments 1-7, wherein the co-modality measurement data is computed tomography (CT) measurement data, and the co-modality image is a CT image.

### Illustrative Embodiment 9

The computer-implemented method of any of illustrative embodiments 1-8, wherein the trained machine learning process is trained on a plurality of histo-images and corresponding co-modality images.

### Illustrative Embodiment 10

The computer-implemented method of any of illustrative embodiments 1-9, comprising:
retrieving machine learning parameters from the data repository, wherein the machine learning parameters characterize the trained machine learning process; and
executing the trained machine learning process based on the machine learning parameters.

### Illustrative Embodiment 11

A non-transitory, computer readable medium storing instructions that, when executed by at least one processor, cause the at least one processor to perform operations comprising:
receiving positron emission tomography (PET) measurement data from an image scanning system;
receiving co-modality measurement data from the image scanning system;
generating a histo-image based on the PET measurement data;
generating a co-modality image based on the co-modality measurement data;
applying a trained machine learning process to the histo-image and the co-modality image and, based on the application of the trained machine learning process to the histo-image and the co-modality image, generating registered attenuation map data characterizing a registered attenuation map; and
storing the registered attenuation map data in a data repository.

### Illustrative Embodiment 12

The non-transitory, computer readable medium of illustrative embodiment 11 storing instructions that, when executed by the at least one processor, further cause the at least one processor to perform operations comprising inputting the histo-image and the co-modality image to a trained neural network.

### Illustrative Embodiment 13

The non-transitory, computer readable medium of illustrative embodiment 12 storing instructions that, when executed by the at least one processor, further cause the at least one processor to perform operations comprising:
generating first feature vectors based on the histo-image;
generating second feature vectors based on the co-modality image; and inputting the first feature vectors and the second feature vectors into the trained neural network.

### Illustrative Embodiment 14

The non-transitory, computer readable medium of any of illustrative embodiments 11-13 storing instructions that, when executed by the at least one processor, further cause the at least one processor to perform operations comprising reconstructing a PET image based on the registered attenuation map data and the histo-image.

### Illustrative Embodiment 15

The non-transitory, computer readable medium of illustrative embodiment 14 storing instructions that, when executed by the at least one processor, further cause the at least one processor to perform operations comprising providing the PET image for display.

### Illustrative Embodiment 16

The non-transitory, computer readable medium of any of illustrative embodiments 11-15 storing instructions that, when executed by the at least one processor, further cause the at least one processor to perform operations comprising generating, by the trained machine learning process, the registered attenuation map data based on detecting a misalignment between the histo-image and the co-modality image.

### Illustrative Embodiment 17

The non-transitory, computer readable medium of illustrative embodiment 16, wherein the detected misalignment is between pixel locations of the histo-image and pixel locations the co-modality image.

### Illustrative Embodiment 18

The non-transitory, computer readable medium of any of illustrative embodiments 11-17, wherein the co-modality measurement data is computed tomography (CT) measurement data, and the co-modality image is a CT image.

### Illustrative Embodiment 19

The non-transitory, computer readable medium of any of illustrative embodiments 11-18, wherein the trained machine learning process is trained on a plurality of histo-images and corresponding co-modality images.

### Illustrative Embodiment 20

The non-transitory, computer readable medium of any of illustrative embodiments 11-19 storing instructions that, when executed by the at least one processor, further cause the at least one processor to perform operations comprising:
retrieving machine learning parameters from the data repository, wherein the machine learning parameters characterize the trained machine learning process; and
executing the trained machine learning process based on the machine learning parameters.

### Illustrative Embodiment 21

A system comprising:
a memory storing instructions; and
at least one processor communicatively coupled to the memory and configured to execute the instructions to:
   receive positron emission tomography (PET) measurement data from an image scanning system;
   receive co-modality measurement data from the image scanning system;
   generate a histo-image based on the PET measurement data;
   generate a co-modality image based on the co-modality measurement data;
   apply a trained machine learning process to the histo-image and the co-modality image and, based on the application of the trained machine learning process to the histo-image and the co-modality image, generate registered attenuation map data characterizing a registered attenuation map; and
   store the registered attenuation map data in a data repository.

### Illustrative Embodiment 22

The system of illustrative embodiment 21, wherein, to apply the trained machine learning process to the histo-image and the co-modality image, the at least one processor is configured to execute the instructions to input the histo-image and the co-modality image to a trained neural network.

### Illustrative Embodiment 23

The system of illustrative embodiment 22, wherein the at least one processor is configured to execute the instructions to:
generate first feature vectors based on the histo-image;
generate second feature vectors based on the co-modality image; and
input the first feature vectors and the second feature vectors into the trained neural network.

### Illustrative Embodiment 24

The system of any of illustrative embodiments 21-23, wherein the at least one processor is configured to execute the instructions to reconstruct a PET image based on the registered attenuation map data and the histo-image.

### Illustrative Embodiment 25

The system of illustrative embodiment 24, wherein the at least one processor is configured to execute the instructions to provide the PET image for display.

### Illustrative Embodiment 26

The system of any of illustrative embodiments 21-25, wherein the at least one processor is configured to execute the instructions to generate the registered attenuation map data based on a detection of a misalignment between the histo-image and the co-modality image.

### Illustrative Embodiment 27

The system of illustrative embodiment 26, wherein the detected misalignment is between pixel locations of the histo-image and pixel locations the co-modality image.

### Illustrative Embodiment 28

The system of any of illustrative embodiments 21-27, wherein the co-modality measurement data is computed tomography (CT) measurement data, and the co-modality image is a CT image.

### Illustrative Embodiment 29

The system of any of illustrative embodiments 21-28, wherein the trained machine learning process is trained on a plurality of histo-images and corresponding co-modality images.

### Illustrative Embodiment 30

The system of any of illustrative embodiments 21-29, wherein the at least one processor is configured to execute the instructions to:
retrieve machine learning parameters from the data repository, wherein the machine learning parameters characterize the trained machine learning process; and
execute the trained machine learning process based on the machine learning parameters.

The apparatuses and processes are not limited to the specific embodiments described herein. In addition, components of each apparatus and each process can be practiced independent and separate from other components and processes described herein.

The previous description of embodiments is provided to enable any person skilled in the art to practice the disclosure. The various modifications to these embodiments will be readily apparent to those skilled in the art, and the generic principles defined herein can be applied to other embodiments without the use of inventive faculty. The present disclosure is not intended to be limited to the embodiments shown herein, but is to be accorded the widest scope consistent with the principles and novel features disclosed herein.

What is claimed is:

1. A computer-implemented method comprising:
receiving positron emission tomography (PET) measurement data from an image scanning system;
receiving co-modality measurement data from the image scanning system;
generating a histo-image based on the PET measurement data;
generating a co-modality image based on the co-modality measurement data;
applying a trained machine learning process to the histo-image and the co-modality image and, based on the application of the trained machine learning process to the histo-image and the co-modality image, generating registered attenuation map data characterizing a registered attenuation map; and
storing the registered attenuation map data in a data repository.

2. The computer-implemented method of claim 1, wherein applying the trained machine learning process to the histo-image and the co-modality image comprises inputting the histo-image and the co-modality image to a trained neural network.

3. The computer-implemented method of claim 2, further comprising:
generating first feature vectors based on the histo-image;
generating second feature vectors based on the co-modality image; and
inputting the first feature vectors and the second feature vectors into the trained neural network.

4. The computer-implemented method of claim 1, further comprising reconstructing a PET image based on the registered attenuation map data and the histo-image.

5. The computer-implemented method of claim 4, further comprising providing the PET image for display.

6. The computer-implemented method of claim 1, wherein the trained machine learning process generates the registered attenuation map data based on detecting a misalignment between the histo-image and the co-modality image.

7. The computer-implemented method of claim 6, wherein the detected misalignment is between pixel locations of the histo-image and pixel locations the co-modality image.

8. The computer-implemented method of claim 1, wherein the co-modality measurement data is computed tomography (CT) measurement data, and the co-modality image is a CT image.

9. The computer-implemented method of claim 1, wherein the trained machine learning process is trained on a plurality of histo-images and corresponding co-modality images.

10. The computer-implemented method of claim 1, comprising:
retrieving machine learning parameters from the data repository, wherein the machine learning parameters characterize the trained machine learning process; and
executing the trained machine learning process based on the machine learning parameters.

11. A non-transitory, computer readable medium storing instructions that, when executed by at least one processor, cause the at least one processor to perform operations comprising:
receiving positron emission tomography (PET) measurement data from an image scanning system;
receiving co-modality measurement data from the image scanning system;
generating a histo-image based on the PET measurement data;
generating a co-modality image based on the co-modality measurement data;
applying a trained machine learning process to the histo-image and the co-modality image and, based on the application of the trained machine learning process to the histo-image and the co-modality image, generating registered attenuation map data characterizing a registered attenuation map; and
storing the registered attenuation map data in a data repository.

12. The non-transitory, computer readable medium of claim 11 storing instructions that, when executed by the at least one processor, further cause the at least one processor to perform operations comprising inputting the histo-image and the co-modality image to a trained neural network.

13. The non-transitory, computer readable medium of claim 12 storing instructions that, when executed by the at least one processor, further cause the at least one processor to perform operations comprising:
generating first feature vectors based on the histo-image;
generating second feature vectors based on the co-modality image; and
inputting the first feature vectors and the second feature vectors into the trained neural network.

14. The non-transitory, computer readable medium of claim 11 storing instructions that, when executed by the at least one processor, further cause the at least one processor to perform operations comprising reconstructing a PET image based on the registered attenuation map data and the histo-image.

15. The non-transitory, computer readable medium of claim 11 storing instructions that, when executed by the at least one processor, further cause the at least one processor to perform operations comprising generating, by the trained machine learning process, the registered attenuation map data based on detecting a misalignment between the histo-image and the co-modality image.

16. A system comprising:
a memory storing instructions; and
at least one processor communicatively coupled to the memory and configured to execute the instructions to:
receive positron emission tomography (PET) measurement data from an image scanning system;
receive co-modality measurement data from the image scanning system;
generate a histo-image based on the PET measurement data;
generate a co-modality image based on the co-modality measurement data;
apply a trained machine learning process to the histo-image and the co-modality image and, based on the application of the trained machine learning process to the histo-image and the co-modality image, generate registered attenuation map data characterizing a registered attenuation map; and
store the registered attenuation map data in a data repository.

17. The system of claim 16, wherein, to apply the trained machine learning process to the histo-image and the co-modality image, the at least one processor is configured to execute the instructions to input the histo-image and the co-modality image to a trained neural network.

18. The system of claim 17, wherein the at least one processor is configured to execute the instructions to:
generate first feature vectors based on the histo-image;
generate second feature vectors based on the co-modality image; and
input the first feature vectors and the second feature vectors into the trained neural network.

19. The system of claim 16, wherein the at least one processor is configured to execute the instructions to reconstruct a PET image based on the registered attenuation map data and the histo-image.

20. The system of claim 17, wherein the at least one processor is configured to execute the instructions to generate the registered attenuation map data based on a detection of a misalignment between the histo-image and the co-modality image.
